(19) United States
(12) Patent Application Publication
(10) Pub. No.: US 2005/0154301 A1
(43) Pub. Date: Jul. 14, 2005

(54) Acoustic diagnosis of sinusitis

(75) Inventors: Menashe Shahar, South Ramat Hagolan (IL); Shahar Katz, Bnei Brak (IL); Yariv Porat, Haifa (IL); Yehuda Holdstein, Haifa (IL); Ori Sahar, South Ramat Hologan (IL)

Correspondence Address: LADAS & PARRY, 26 WEST 61ST STREET, NEW YORK, NY, 10023, US

(73) Assignee: ACOUSTITECH LTD.

(21) Appl. No.: 11/005,315

(22) Filed: Dec. 6, 2004

Related U.S. Application Data
(63) Continuation-in-part of application No. PCT/IL03/00397, filed on May 15, 2003.

(30) Foreign Application Priority Data
Jun. 12, 2002 (IL) 150189

(51) Int. Cl. A61B 8/00

(52) U.S. Cl. 600/437

(57) ABSTRACT

A system for diagnosing sinusitis in a human subject including: a miniature transducer for emitting a sound signal; a miniature microphone for detecting sound signals; a transducer holder configured to contain the miniature transducer and a microphone holder configured to contain the miniature microphone, each configured to be inserted into a nostril of a human subject; signal processing apparatus for controlling sound signals emitted by the miniature transducer and for processing sound signals detected by the miniature microphone; and data analysis apparatus for analyzing the processed detected sound signals, which includes a data storage device for storing processed sound signals, baseline signals representing measurements taken on healthy and variously-infected subjects, and programs for data analysis for reaching a diagnosis.

### FIELD OF THE INVENTION

The present invention relates generally to the diagnosis of sinusitis. More particularly, the present invention relates to an acoustic means and to a method used for diagnosing sinusitis using said acoustic means.

### BACKGROUND OF THE INVENTION

Rhinometry, the measurement of the nasal region, is a known field with a number of medical implications. One means of measurement employed is acoustic, in which sound waves are used as the probing energy.

U.S. Pat. No. 5,666,960 discloses a method and device for performing measurement of the respiratory tract. The analytical method in U.S. Pat. No. 5,666,960 is reflectometry. A burst of sound pulses (or a single pulse), the nature of which is not disclosed, is emitted into the nose to probe nasal morphology, by forming what, may be called, an “acoustic image” of the nose space. The device disclosed in U.S. Pat. No. 5,666,960 is a tube plunged into the nose, which has a loudspeaker on one end, and several microphones on the side of the tube, at prescribed intervals. The tube is quite long and the device of U.S. Pat. No. 5,666,960 cannot provide the morphology of the sinuses, as it is intended, and therefore adapted exclusively, for probing air flow and respiration.

U.S. Pat. No. 5,848,973 discloses is a device that is an adaptation of the device of U.S. Pat. No. 5,666,960, where the device includes a mechanism filter to avoid contamination, and U.S. Pat. No. 5,902,237 discloses an improved method which involves synchronization of the emitted burst of pulses to the respiratory rhythm for improving the accuracy of the method. Being in synchronization with the respiratory rhythm implies that the application is respiration oriented and not rhinometry oriented.

U.S. Pat. No. 5,823,965 discloses using a method similar to the method of U.S. Pat. No. 5,666,960 to examine air passages in a biological subject. Here too, the emphasis is put on airflow in such passages. U.S. Pat. No. 5,823,965 does not mention sinuses or measurement procedure in that respect.

It is therefore an object of the present invention to provide a system and a method for examining the system of nasal cavities in a human subject, including the nasal sinuses, particularly in order to diagnose sinusitis, using acoustic means.

It is another object of the present invention to provide a device and a method for examining the system of nasal cavities in a human subject, which is minimally invasive and cause minimal discomfort to the subject or patient being so examined.

It is yet another object of the present invention to provide a system and a method for examining other systems of body cavities in a mammalian subject to diagnose pathological conditions therein.

Other objects and advantages of the invention will become apparent as the description proceeds.

### SUMMARY OF THE INVENTION

The present invention provides a system for examining the system of nasal cavities in a human subject, including the nasal sinuses, particularly in order to diagnose sinusitis, comprising:
a) A first sound emitter, housed within a first holder that is adapted to be slideably and sealingly inserted into nostrils of a human subject;
b) A first sound detector, housed within a second holder that is adapted to be slideably and sealingly inserted into nostrils of a human subject;
c) A transmission/recording controller, connected to said first sound emitter, to cause it to emit a predetermined acoustic wave whenever desired, and to said first sound detector, for recording acoustic reflections;
d) A signal processing apparatus with dedicated software, for: (i) controlling the operation of the transmission/recording controller and, thereby, the acoustic wave emitted from said first sound emitter, and (ii) processing detected acoustic waves; and
e) Data analysis apparatus, for analyzing the processed acoustic wave, the analysis apparatus including a data analysis portion with dedicated software for performing the analysis, and a data storage array for storing at least: (i) data relating to emitted and processed acoustic waves, (ii) data relating to mathematical model(s) useful in interpreting the processed acoustic waves, and, optionally, (iii) fiducial reference data, to which data relating to the processed acoustic waves is, whenever desired, compared;
   wherein,
A predetermined acoustic wave is emitted from the first sound emitter through a first, and/or a second, nostril(s) of the human subject, whereby to generate a first, and, where relevant, a second reflected acoustic wave(s) that is/are detected by the first sound detector through the second, and/or first, nostril(s), respectively. Then, the first detected acoustic wave is processed and analyzed to reach a diagnosis or, in case two predetermined acoustic waves are emitted, each time through a different nostril and possibly at the same time, the two generated reflected acoustic waves are processed and the differences therebetween are analyzed to reach a diagnosis.

According to a preferred embodiment of this invention, the first and the second holders are the same holder (hereinafter “first double holder”) and the system further comprises a second sound emitter and a second sound detector, both housed within a second “double holder”. By “double holder” is meant a holder that both houses a sound emitter and a sound detector, and is adapted to be slideably and sealingly inserted into a nostril. According to this embodiment, the first and second double holders are each inserted into a different nostril. Then, first and second predetermined acoustic waves (which can have the same, or different, characteristics) are emitted, each signal through a different nostril and possibly at the same time, and the resulting reflections are detected through the corresponding nostril. The detected acoustic reflections are processed and analyzed as described hereinbefore.

According to a preferred embodiment, the analysis further comprises a step of comparing the data relating to the processed acoustic waves to the fiducial reference data and reaching diagnosis based on the comparison.

The fiducial reference data characterizes typical healthy and variously sinusitis-infected human subjects, and it can be derived theoretically and/or empirically using a suitable mathematical model and employing a conventional technology. Such a conventional technology may be, for example, a technology widely referred to as Computerized Tomography (CT). In addition, the fiducial reference data may further include data relating to volumes of sinusitis, or it may include data that assists in the calculation of such volumes.

According to another preferred embodiment, the analysis further comprises calculation of the volume of the sinuses of the subject by mathematically modeling the sinuses, for example, as Helmholtz Resonators. Of course, other mathematical models may be employed as well.

The emitted acoustic waves can be of any type suitable for the purposes of this invention, and, preferably, they are selected from the group consisting of {‘White noise’ pulses; chirp sound pulses of a particular frequency range; any desired frequency sweep in some predetermined frequency range; Amplitude Modulated (AM) signal; Frequency Modulated (FM) signal; a train of AM signal as a function of time; a train of FM signal as a function of time}. Of course, different signals may be utilized to accomplish the purposes of this invention, which depend on the specific hardware components of the system and on the actual mathematical model employed.

Preferably, the acoustic waves are emitted through a nostril to the nasal cavities of the human subject subsequent to the object inhaling and immediately thereafter sealing his nasal cavities internally for a time period sufficient (e.g. normally 2 to 5 seconds) for carrying out a measurement cycle that consists of emission of the acoustic wave and detection of the resulting acoustic reflection.

The system may include means, such as a display and a printer, for presenting to a therapist, in any convenient way, any desired data concerning the analysis of the detected acoustic waves, and, in particular, the resulting diagnosis.

The present invention also provides a method for examining the system of nasal cavities in a human subject, including the nasal sinuses, particularly in order to diagnose sinusitis, comprising:
a) Emitting, from a first sound emitter, a first predetermined acoustic wave through a first nostril into the nasal cavities of the subject;
b) Detecting, by a first sound detector and through the second nostril, a first reflected acoustic wave resulting from the emitted acoustic wave; and
c) Processing and analyzing the first detected acoustic reflection to diagnose sinusitis in the subject.

Preferably, the method further comprises:
a) Emitting, from the first sound emitter housed within a first holder and through the second nostril, a second predetermined acoustic wave;
b) Detecting, by the first sound detector housed within a second holder and through the first nostril, a second reflected acoustic wave resulting from the emission of the second acoustic wave;
c) Processing the second detected acoustic reflection; and
d) Analyzing the second detected acoustic reflection and the differences between the first and second reflected acoustic reflections to diagnose sinusitis in the subject.

According to a preferred embodiment of this invention, the first and the second holders are the same holder (hereinafter “first double holder”) and the method further comprises using a second sound emitter and a second sound detector, both housed within a second “double holder”. According to this embodiment, the first and second double holders are each inserted into a different nostril. Then, first and second predetermined acoustic waves (which can have the same, or different, characteristics) are emitted, each signal through a different nostril and possibly at the same time, and the resulting reflections are detected through the corresponding nostril. The detected acoustic reflections are processed and analyzed as described hereinbefore.

According to a preferred embodiment, the analysis further comprises a step of comparing the data relating to the processed acoustic waves to pre-stored fiducial reference data, and reaching diagnosis based on the comparison.

According to another preferred embodiment, the analysis further comprises calculation of the volume of the sinuses of the subject by mathematically modeling the sinuses, for example, as Helmholtz Resonators. Of course, other mathematical models can be employed as well.

The emitted acoustic waves can be of any type suitable for the purposes of this invention, and, preferably, they are selected from the group consisting of {‘white noise’ pulses; chirp sound pulses of a particular frequency range; any desired frequency sweep in some predetermined frequency range; Amplitude Modulated (AM) signal; Frequency Modulated (FM) signal; a train of AM signal as a function of time; a train of FM signal as a function of time}. Of course, different signals may be utilized to accomplish the purposes of this invention, which depend on the specific hardware components of the system and on the actual mathematical model employed.

Preferably, the acoustic waves are emitted through a nostril to the nasal cavities of the human subject subsequent to the object inhaling and immediately thereafter sealing his nasal cavities internally for a time period sufficient (e.g. normally 2 to 5 seconds) for carrying out a measurement cycle that consists of emission of the acoustic wave and detection of the resulting acoustic reflection.

The system may include presentation means, such as a display and a printer, for presenting to a therapist, or to another person, in any convenient way, any desired data concerning the analysis of the detected acoustic waves, and, in particular, the resulting diagnosis.

According to another aspect of the invention, the analysis of the detected reflection(s) further includes using statistical tools for determining whether the subject is sinusitis-infected, and, if the subject is determined as such, for determining the severity of the infection.

In a preferred embodiment of this invention, analyzing the detected acoustic reflections involves directly resolving Acoustic Wave Equation(s) that characterize the detected acoustic reflections, using proper boundary conditions.

The system and method disclosed in the present invention can be utilized, mutatis mutandis, for diagnosing pathological conditions in body cavities in a mammalian subject.

The system and method disclosed in the present invention can be utilized, mutatis mutandis, for diagnosing pathological conditions in body cavities such as the lungs and their associated air passages, and chambers of the heart.

### BRIEF DESCRIPTION OF THE DRAWINGS

In the drawings:

FIG. 1 schematically illustrates the nasal cavity, the nostrils and associated sinuses together with a block diagram of a system for diagnosing sinusitis, according to a preferred embodiment of the present invention.

### DETAILED DESCRIPTION OF PREFERRED EMBODIMENTS

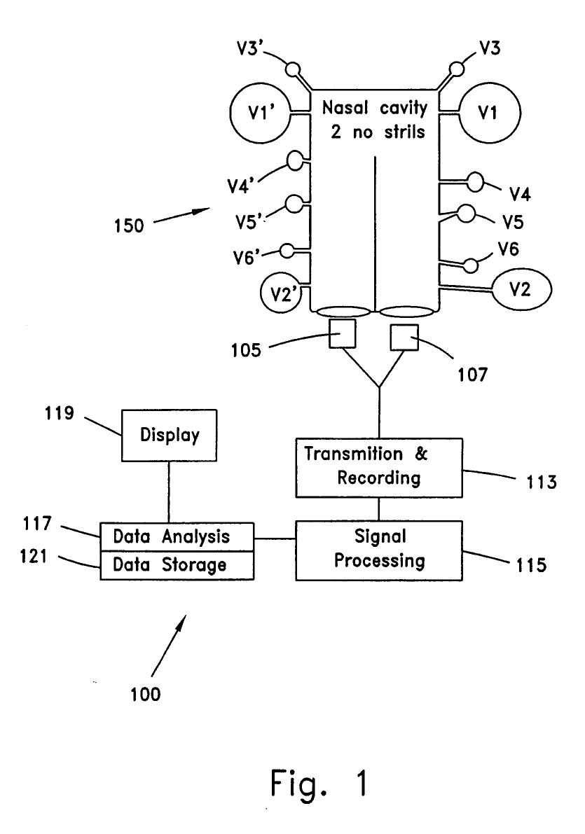

Referring now to FIG. 1, there is shown a schematic representation of the nasal cavity, the nostrils, and associated sinuses, referred to generally as 150, together with a block diagram of a system, referred to generally as 100, for diagnosing sinusitis, constructed and operative in accordance with a preferred embodiment of the present invention.

System 100 includes a first miniature transducer (sound emitter) 105 and a first miniature microphone (sound detector) 107 for emitting and detecting, respectively, acoustic waves in the nasal cavities of a human subject. Transducer 105 and microphone 107 are fitted in a first and second holders (not shown), respectively, that allow them to be slideably and sealingly inserted into the nostrils of the subject. They are connected to an acoustic transmission and recording unit 113, which controls the acoustic waves emitted by transducer 105 and records acoustic waves detected by microphone 107. Transmission and recording unit 113 is, in turn, connected to signal processing unit 115 which prepares the detected acoustic waves for use by data processing and analyzing unit 117, which performs data processing and analysis on processed detected acoustic waves.

Data processing and analysis unit 117 includes a data storage device 121 for storing data relating to emitted acoustic waves, processed acoustic waves and baseline signals representing measurements taken on healthy subjects, as well as programs for data analysis, and may also have an associated presentation device 119, for example a display, for presenting to a therapist, or operator of the system, data of interest, such as results of measurements and analysis, and final diagnosis.

Alternatively, the first sound emitter 105 and the first sound detector can be housed within a first ‘double holder’ and additional, substantially identically structured, second double holder may be used, having fitted therein both a transducer, such as transducer 105, and a microphone, such as microphone 107. The first and second double holders may be slideably and sealingly inserted into the nostrils of the subject, each double holder into a different nostril, in accordance with an alternative embodiment of the present invention.

Transmission and recording unit 113 can then selectively activate a transducer 105 and microphone 107 combination to generate, for detection, reflected acoustic waves for different experimental configurations without hassling the subject with rearranging transducer 105 and microphone 107 holders.

According to one preferred embodiment of the pressure invention, the method includes the steps of:
inserting transducer 105, while in its holder, into one nostril of a subject and microphone 107, while in is holder, into the second nostril of the subject, thereby externally sealing off the nostrils of the subject;
subsequent to the subject inhaling and immediately thereafter internally sealing his nasal cavities, emitting, via transducer 105 in the first nostril of the subject, a first predetermined probe acoustic wave into the nasal cavities 150 of the subject. The step of inhaling and internally sealing of the nasal cavities is not necessary but, rather, it is only an option;
detecting, via microphone 107 in the second nostril of the subject, reflected acoustic waves from the nasal cavities 150 of the subject in response to the emission of the first predetermined probe acoustic wave;
subsequent to the subject again inhaling and immediately thereafter sealing his nasal cavities internally, emitting, via transducer 105 in the second nostril of the subject, a second predetermined probe acoustic wave into the nasal cavities 150 of the subject;
detecting, via microphone 107 in the first nostril of the subject, reflected acoustic waves from the nasal cavities 150 of the subject in response to the emission of the second predetermined probe acoustic wave;
comparing two sets of detected acoustic waves after both sets are prepared, by transmission and recording unit 113 and signal processing unit 115, for analysis via data processing and analysis unit 117; and
analyzing, via data processing and analysis unit 117, the two sets of detected acoustic waves and the differences therebetween to diagnose sinusitis in the subject.

As described hereinbefore in accordance with another preferred embodiment of the present invention, diagnosing sinusitis can be implemented by emitting only the first predetermined probe acoustic wave, and detecting and analyzing the resulting acoustic reflections. Emitting a second probe acoustic wave and detecting, as a result of this emission, a second acoustic reflection may normally result in a more enhanced diagnosis results, though the difference may prove to be uncritical.

In cases where two predetermined probe acoustic waves are utilized, they can be emitted, and their corresponding acoustic reflections detected, by physically switching two holders between the nostrils of the subject, one holder fitted with a transducer 105 and another holder fitted with a microphone 107.

Alternatively, when two substantially identical double holders are employed, each having fitted therein both a transducer 105 and a microphone 107, transmission and recording unit 113 selectively activates transducers 105 and microphones 107 to generate respective acoustic waves/reflections without hassling the subject with the rearrangement of the (transducer 105 and microphone 107) holders.

The probe acoustic waves emitted may be sound pulses of particular frequencies or they may be ‘white noise’ pulses, chirp pulses of a particular frequency range, AM or FM modulated periodic signals or a train of such signals as a function of time, or some other frequency sweep in some predetermined range, preferably within the frequency range of 100 Hz to 20 kHz. Pulse length is typically 1 to 20 seconds, and the sound intensity level is preferably 70 dB to 60 dB.

The volume or the acoustic impedance of the nasal cavities 150 can be obtained by comparing the detected acoustic waves to baseline signals that may be based on theoretical modeling of the nasal cavities 150 or on actual measurements taken on healthy and variously sinusitis-infected subjects. In the case of the nasal cavities, the calculation is based on modeling the system of nasal cavities and sinuses as, for instance, one or more Helmholtz resonators, as is explained hereinbelow. Variations in the measured volume showing reduction from the baseline volume can indicate a blockage symptomatic of sinusitis. The extent of the blockage indicates the severity of the sinusitis.

The modeling of the nasal and paranasal volumes as a set of connected Helmholtz resonators is described hereinafter only to exemplify employment of a mathematical model to characterize cavities in a human subject. However, any person skilled in the art may employ other models. In addition, cavities can be characterized by directly resolving related acoustic wave equation(s) using proper boundary conditions. However, for the sake of simplicity the nasal cavities and the sinuses are represented as a collection of connected chambers, each of which is modeled as a separate Helmholtz resonator having a characteristic primary resonant frequency that depends on its volume, linear dimensions, and the elastic properties of its internal surfaces. The overall connected system acts as a band pass filter with its own characteristic frequencies. The relevant equations for the characteristic frequencies are given by:
V₀=(1/2π)*{square root over ((c²)}S/IₑV); and (1)
V₀=(1/π)*{square root over ((c²)}S/IₑVₚ) (2)
where
V₀—Helmholtz resonator frequency;
S—area of each port in the nose;
VP—partial adapter volume=adapter volume divided by ‘n’ (number of ports);
V—Helmholtz resonator volume;
Iₑ—≅1+0.8*\sqrt{S};
l—length of port;
c—sound speed in air; and
ω—frequency.

Referring again to FIG. 1, the effective volume V_{E} of the nasal cavity, the nostrils and the associated sinuses 100 may be expressed as:
1/V_{E}=1/V_{N}+1/V_{Si}+1/V_{Si′}+, . . . , (3)
where
V_{N} is the volume of the nasal cavity and the nostrils, and
V_{Si} and V_{Si′} are volumes of sinuses pairs (v1,v1′; v2, v2′, etc.).

As will be understood by those skilled in the art, if one of a pair of sinuses or the passage thereto is blocked by sinusitis, the effective volume, and therefore the resonant frequencies, will change accordingly. By employing, for instance, Fast Fourier Transform (FFT) on the detected acoustic waves, their frequencies can be calculated and compared to expected values.

For example, in a typical adult, the frequency for the nasal cavity is 211 Hz and for each maxillary sinus, which are normally very close in size, will be 308 Hz. Ignoring the effect of the other, smaller, sinuses, the combined system will have a characteristic frequency of 969 Hz. If one of the sinuses is blocked, the characteristic frequency will be 747 Hz. In cases where both sinuses are blocked, the characteristic frequency will revert to the value for the nasal cavity alone. As will be understood by those skilled in the art, frequency differences such as these are readily resolvable.

It should be noted that other methods may be utilized for analyzing nasal and paranasal cavities in a human subject. In particular, a direct, or indirect, analytical or numerical solution of the related acoustical wave equation may be used to model the effective volumes that may be affected by sinusitis, while proper boundary conditions are taken into account.

Is noted that included in the scope of the present invention are embodiments for examining other sub-systems in human and in other mammalian subjects, such as cattle. Basically, all that is needed is adapting the transducer and microphone holders to the sub-system of interest, and corresponding modifications in the processing and analysis software. For example, with suitable arrangement for emitting and detecting acoustic waves, the lungs and their air passages, or the heart chambers, may be examined for abnormal or pathological conditions using the system and method of the present invention.

The above embodiments have been described by way of illustration only and it will be understood that the invention may be carried out with many variations, modifications and adaptations, without departing from its spirit or exceeding the scope of the claims.

1. System for examining the system of nasal cavities in a human subject, including the nasal sinuses, particularly in order to diagnose sinusitis, comprising:
a) a first sound emitter, housed within a first holder that is adapted to be slideably and sealingly inserted into nostrils of human subjects;
b) a first sound detector, housed within a second holder that is adapted to be slideably and sealingly inserted into nostrils of human subjects;
c) a transmission and recording controller, connected to said first sound emitter, to cause it to emit a predetermined acoustic wave whenever desired, and to said first sound detector, for recording acoustic reflections;
d) a signal processing apparatus with dedicated software, for: (i) controlling the operation of said transmission and recording controller and, thereby, the acoustic wave emitted from said first sound emitter, and (ii) processing detected acoustic waves; and
e) data analysis apparatus, for analyzing the processed acoustic wave, the analysis apparatus including a data analysis portion with dedicated software for performing the analysis, and a data storage array for storing at least: (i) data relating to emitted and processed acoustic waves, (ii) data relating to mathematical model(s) useful in interpreting the detected acoustic waves, and, optionally, (iii) fiducial reference data, to which data relating to the processed acoustic waves is, whenever desired, compared;
wherein,
said predetermined acoustic wave is emitted from the first sound emitter through a first, and/or a second, nostril(s) of said subject, whereby to generate a first and, where relevant, a second reflected acoustic wave(s) that is/are detected by said first sound detector through said second, and/or first, nostril(s), respectively; said first detected acoustic wave being processed and analyzed to reach a diagnosis or, in case two predetermined acoustic waves are emitted, each time through a different nostril, the two generated reflected acoustic waves being processed and analyzed and the differences therebetween being analyzed to reach a diagnosis.

2. System according to claim 1, wherein the first and the second holders are the same holder, being a ‘first double holder’, and said system further comprising a second sound emitter and a second sound detector, both housed within a second double holder and connected to the transmission and recording controller, said first and second double holders are each inserted into a different nostril,
wherein,
a first and second predetermined acoustic waves, having the same or different characteristics, are emitted, each wave through a different nostril and possibly at the same time, and the acoustic reflections resulting from their emission being detected through the corresponding nostril and analyzed to reach a diagnosis.

3. System according to claim 1, wherein the analysis comprising a step of comparing the data relating to the processed acoustic waves to the fiducial reference data and reaching diagnosis based on the comparison.

4. System according to claim 3, wherein the analysis further comprises calculation of the volume of the sinuses of the subject by mathematically modeling the sinuses.

5. System according to claim 3, wherein the fiducial reference data characterizes typical healthy and variously sinusitis-infected human subjects, said data being derived theoretically and/or empirically using a suitable mathematical model and employing one or more conventional technologies, said data also including data relating to volumes of sinusitis, and/or data assisting in the calculation of said volumes.

6. System according to claim 1, wherein the emitted acoustic waves are selected from the group consisting of {‘white noise’ pulses; chirp sound pulses of a particular frequency range; any desired frequency sweep in some predetermined frequency range; Amplitude Modulated (AM) signal; Frequency Modulated (FM) signal; a train of AM signal as a function of time; a train of FM signal as a function of time}.

7. System according to claim 1, wherein, the acoustic waves are emitted through a nostril to the nasal cavities of the human subject subsequent to said object inhaling and immediately thereafter sealing his nasal cavities internally for a time period sufficient for carrying out a measurement cycle consisting of emission of said acoustic waves and detection of the resulting acoustic reflection.

8. System according to claim 1, further comprising means for presenting to a therapist, or to another person, in any convenient way, any desired data concerning the analysis of the detected acoustic waves and the resulting diagnosis.

9. System according to claim 8, wherein the presenting means is a display and/or a printer.

10. System according to claim 1, adapted for diagnosing various types of body cavities of a human subject and other mammalian.

11. System according to claim 4, wherein the mathematical model is the Helnholtz Resonator model.

12. System according to claim 1, wherein the analysis involves directly resolving Acoustic Wave Equation(s) characterizing the detected acoustic reflections, using proper boundary conditions.

13. Method for examining the system of nasal cavities in a human subject, including the nasal sinuses, particularly in order to diagnose sinusitis, comprising:
a) emitting, from a first sound emitter and through a first nostril of said subject, a first predetermined acoustic wave into the nasal cavities of said subject;
b) detecting, by a first sound detector and through the second nostril, a first reflected acoustic wave resulting from the emission of said acoustic wave; and
c) processing and analyzing the first detected acoustic reflection to diagnose sinusitis in said subject.

14. Method according to claim 13, further comprising:
a) emitting, from the first sound emitter housed within a first holder, a second predetermined acoustic wave through the second nostril;
b) detecting, by the first sound detector housed within a second holder and through the first nostril, a second reflected acoustic wave resulting from the emission of said second predetermined acoustic wave;
c) processing said second detected reflection; and
d) analyzing said second detected acoustic reflection and the differences between the first and said second acoustic reflections to diagnose sinusitis in the subject.

15. Method according to claim 13, wherein the first and the second holders are the same holder, being a ‘first double holder’, and the method further comprising a step of using a second sound emitter and a second sound detector, both housed within a second ‘double holder’, said first and second double holders are each inserted into a different nostril, and a first and second predetermined acoustic waves, having the same, or different, characteristics, are emitted, each through a different nostril and possibly at the same time, and the resulting reflections being detected through the corresponding nostril, the detected acoustic reflections being processed and analyzed to reach a diagnosis.

16. Method according to claim 13, wherein the analysis comprising a step of comparing the data relating to the processed acoustic waves to pre-stored fiducial reference data, and reaching diagnosis based on the comparison.

17. Method according to claim 16, further comprising a step of calculating the volume of the sinuses of the subject by mathematically modeling the sinuses.

18. Method according to claim 13, wherein the emitted acoustic waves are selected from the group consisting of {‘white noise’ pulses; chirp sound pulses of a particular frequency range; any desired frequency sweep in some predetermined frequency range; Amplitude Modulated (AM) signal; Frequency Modulated (FM) signal; a train of AM signal as a function of time; a train of FM signal as a function of time}.

19. Method according to claim 18, wherein the acoustic waves are emitted through a nostril to the nasal cavities of the human subject subsequent to the object inhaling and immediately thereafter sealing his nasal cavities internally for a time period sufficient for carrying out a measurement cycle consisting of emission of the acoustic waves and detection of the resulting acoustic reflections.

20. Method according to claim 13, wherein any desired data concerning the analysis of the detected acoustic waves and the resulting diagnosis is presented to a therapist, or to other person, in any convenient way.

21. Method according to claim 20, wherein the presentation is implemented using a display and/or a printer.

22. Method according to claim 16, wherein the analysis of the detected reflection(s) further comprises a step of using statistical tools for determining whether the subject is sinusitis-infected, and, if said subject is determined as such, for determining the severity of the infection.

23. Method according to claim 13, adapted for diagnosing various types of body cavities of a human subject and other mammalian.

24. Method according to claim 17, wherein the mathematical model is the Helmholtz Resonator model.

25. Method according to claim 13, wherein the analysis involves directly resolving Acoustic Wave Equation(s) characterizing the detected acoustic reflections, using proper boundary conditions.
